(19) United States
(12) Patent Application Publication
(10) Pub. No.: US 2006/0293581 A1
(43) Pub. Date: Dec. 28, 2006

(54) Marker device for X-ray, ultrasound and MR imaging

(75) Inventors: Donald B. Plewes, Toronto (CA); Yangmei Li, North York (CA); Jian-xiong Wang, Guelph (CA)

Correspondence Address: Mark A. Litman & Associates, P.A., York Business Center, Suite 205, 3209 West 76th St., Edina, MN, 55435, US

(73) Assignee: Sunnybrook and Women's College Health Sciences Centre

(21) Appl. No.: 11/128,013

(22) Filed: May 12, 2005

(51) Int. Cl. A61B 5/05

(52) U.S. Cl. 600/407

(57) ABSTRACT

An imaging marker comprised of glass and iron-containing aluminum microspheres in a gel matrix which shows uniformly good contrast with MR, US and X-Ray imaging. The marker is small and can be easily introduced into tissue through a 12-gauge biopsy needle. The concentration of glass microspheres and the size dictate the contrast for US imaging. The contrast seen in MRI resulting from susceptibility losses is dictated by the number of iron-containing aluminum microspheres; while the artifact of the marker also depends on its shape, orientation and echo time. By optimizing the size, iron concentration and gel binding, an implantable tissue marker is created which is clearly visible with all three imaging modalities.

### BACKGROUND OF THE INVENTION

1. Field of the Invention

The present invention relates to the field of medical imaging, in particular to imaging procedures that utilize implantable markers for localizing, identifying, and treating abnormal tissues in the human body under each of X-ray, ultrasound (US), and magnetic resonance imaging (MRI) guidance.

2. Background of the Art

Breast tissue conserving surgical methods are increasingly being used for tumor resection in part because of significant improvements in imaging detection of small node-negative breast tumors. Accurate localization and identification of the spatial extent of a tumor is highly desirable in pre-operative surgical planning to minimize damage to normal tissues while at the same time ensuring that the tumor is entirely removed. Guidewire markers are the most commonly used device for pre-operative localization of breast lesions performed under X-ray mammography and US imaging, and more recently under MRI, as reported in the medical literature by Makoske et al (Makoske T, et al., 2000 Am Surg 66: 1104-8), Staren and O'Neill (Staren E D and O'Neill T P 1999 Surgery 126: 629-34), Bedrosian et al (Bedrosian I, et al., 2003 Cancer 98; 468-73 Bedrosian I, et al., 2002 Ann Surg Oncol 9; 457-61), and Warner et al (Warner E, et al., 2001 J Clin Oncol 19: 3524-31). Once positioned, the guidewire marker is intended to enable a surgeon to pre-operatively establish tumor margins or biopsy sites by reference to the position of the marker. Surgeons typically use US to localize the guidewire marker in relation to associated tissue lesions. Exemplary of traditional needle localized markers for breast biopsy and surgery procedures is U.S. Pat. No. 6,181,960 (Jensen et al.) which discloses a radiographic marker comprised of a single piece of wire folded to form the limbs and shaft of an arrow which can be directed to point to a specific site in a tissue.

Published studies, for example, Rissanen et al (Rissanen T J, et al., 1993 Clin Radiol 47: 14-22), have shown that the US visibility of guidewire markers currently used in breast tumor localization is suboptimal in 4-9% of surgical cases. Furthermore, transdermal placement of the guidewire has been reported to result in adverse vasovagal reactions in 10-20% of patients (Rissanen et al. supra, Ernst et al. (Ernst M F, et al., 2002 Breast 11; 408-13), Abrahamson et al. (2003 Acad Radiol 10; 601-6), Jackman and Marzoni (Jackman R J and Marzoni F A, 1997 Radiology 204; 677-84). A second adverse effect of transdermal placement of guidewire markers is that placement of the guidewire and the surgical procedure generally must be completed within the same day. This necessitates significant scheduling challenges between the departments of surgery and radiology and may even compromise the health of the patient in some instances.

Ideally, applicants have determined that a marker used for imaging localization of tumors and other lesions should be visible with all three imaging modalities. While this is not a problem for mammography, currently used guidewire markers can obscure the visibility of tissue lesions due to large and uncontrolled magnetic susceptibility artifacts arising from the material of fabrication. Magnetic susceptibility is a quantitative measure of a material's tendency to interact with and distort an applied magnetic field. This effect makes verification of accurate localization difficult and can degrade the quality of the diagnostic information obtained from the image. Localization markers used in MRI should therefore be MR-compatible in both static and time-varying magnetic fields. Although the mechanical effects of the magnetic field on ferromagnetic materials present the greatest danger to patients because of possible unintended movement of the guidewire, it is also possible that tissue and device heating may result from radio-frequency power deposition in electrically conductive material present within the imaging volume. Any material that is added to the structure of a marker to improve its MR visibility must not contribute significantly to its overall magnetic susceptibility, or imaging artifacts could be introduced during the MR process. Image distortion may generally include local or regional signal loss, signal enhancement, or altered background noise. Applicants have found that markers used in tumor localization should also be made of material that is temporally stable so as to ensure reliable contrast, mechanically stable to ensure mechanical integrity, and tissue compatible.

Initial strategies to position and visualize implantable devices used in MRI-guided procedures were based on passive susceptibility artifacts produced by the devices when exposed to the MR field. U.S. Pat. No. 4,827,931, Longmore) and U.S. Pat. Nos. 5,154,179 and 4,989,608 (Ratner) disclose the incorporation of paramagnetic material into medical devices such as catheters to make the devices visible under MR imaging. U.S. Pat. No. 5,211,166 (Sepponen) similarly discloses the use of surface impregnation of various “relaxants,” including paramagnetic materials and nitrogen radicals, onto surgical instruments to enable their MR identification. However, these inventions do not provide for artifact-free MR visibility in the presence of rapidly alternating magnetic fields, such as would be produced during high-speed MR imaging procedures. The magnetic susceptibility artifact produced by the marker during MRI exams must be small enough not to obscure surrounding anatomy, or mask low-threshold physiological events that have an MR signature, which could compromise the surgeon's ability to perform the intervention. Consequently, guidewire markers and other implantable devices positioned within the MR imager must be made of materials that have properties compatible with their use in human tissues during MR imaging procedures, including real-time MR imaging. An improved method for passive MR visualization of implantable medical devices is disclosed in U.S. Pat. No. 5,744,958 (Werne), wherein an ultra thin coating of conductive material is applied such that the susceptibility artifact due to the metal is negligible due to the low material mass. At the same time, the eddy currents associated with the device are limited because of the ultra-thin conductor coating. A similar method employing a nitinol wire with Teflon® coat, in combination with extremely thin wires of a stainless steel alloy included between the nitinol wire and Teflon® coat, has been reported in the medical literature by Frahm et al. (Frahm et al., 1997 Proc. ISMRM 3: 1931).

Exemplary of methods for active MR visualization of implantable medical devices are U.S. Pat. No. 5,211,165 (Dumoulin et al.), U.S. Pat. Nos. 6,026,316 and 6,061,587 (Kucharczyk and Moseley), U.S. Pat. No. 6,272,370 (Gillies et al.), and U.S. Pat. No. 6,626,902 (Kucharczyk and Gillies). These inventions disclose MR tracking systems based on transmit/receive radiofrequency coils positioned near the end of an implantable medical device by which the position and orientation of the device can be localized using radio frequency field gradients. MRI-guided procedures using active visualization of implantable medical devices have also been described in the medical literature, for example, by Hurst et al. (Hurst et al., 1992 Mag Res Med 24: 343-357), Kantor et al. (Kantor et al., 1984 Circ. Res 55: 55-60), Kandarpa et al. (Kandarpa et al., 1991 Radiology 181: 99), Bornert et al. (Bornert et al., 1997 Proc. ISMRM 3: 1925), Coutts et al. (Coutts et al., 1997 Proc. ISMRM 3: 1924), Wendt et al. (Wendt et al., 1997 Proc ISMRM 3: 1926), Langsaeter et al. (Langsaeter et al., 1997 Proc. ISMRM 3: 1929), Zimmerman et al. (Zimmerman et al., 1997 Proc. ISMRM 3: 1930), and Ladd et al. (Ladd et al., 1997 Proc. ISMRM 3: 1937).

The limitations of guidewire markers for imaging localization of breast tumors have prompted alternative approaches. For example, Bargaz (Bergaz F, et al., 2002 Eur Radiol 12 471-4) has reported the use of a 3 mm stainless steel clip which is released with a specialized applicator and is clearly visible by mammography. However, these clips can migrate over time, limiting their accuracy for excisional biopsy procedures (Birdwell and Jackman, 2003 Radiology 229; 541-4). Fajardo (Fajardo L L, et al., 1998 Radiology 206; 275-8) has described the use of an endovascular embolization coil which can be deployed in tissue through a biopsy needle and has good mammographic visualization and stability over a 6 month period. Harms (Harms S E, et al., 2002 ISMRM 11: 633) has demonstrated the utility of a small hematoma as an MRI marker by injecting the patient's blood near the tumour mass. U.S. Pat. No. 6,714,808 (Klimberg et al.) further discloses a method of hematoma-directed US guided excisional breast biopsy, wherein the hematoma is produced by an injection of the patient's own blood into a pre-selected area to target a lesion. Unlike the present invention, however, none of the markers reported in the prior art are clearly visible under X-ray, U.S. and MRI and can be used to guide MRI, X-ray, and US-guided surgical and biopsy procedures in any region of the body. There is therefore a need for a single non-migrating tissue compatible imaging marker that is reliably and conspicuously visible on X-ray, US and MRI without any degradation in the diagnostic quality of the images.

### SUMMARY OF THE INVENTION

The present invention provides a novel interstitial marker comprised of [ceramic, various metals, or plastics] glass or copper and aluminum microspheres in a gel matrix which marker shows uniformly good contrast with each of magnetic resonance (MR), Ultrasound (US) and X-Ray imaging. The marker is small and can be easily introduced into tissue through a 12-gauge biopsy needle. The concentration and size of the microspheres determine the contrast for US imaging. The contrast seen on MRI resulting from induced magnetic susceptibility is determined by the number of iron-containing aluminum microspheres added to the marker, the shape and orientation of the marker, and the echo time of the MRI pulse sequence. By selecting materials of a range of atomic numbers and density higher than that of biological tissues, the x-ray attenuation coefficients of the constituent materials in the marker also provide clear visualization via x-ray imaging.

By optimizing the size, iron concentration and gel binding, a marker can be created which is clearly visible with all three imaging modalities. The marker disclosed in this invention overcomes numerous limitations of currently used imaging localization devices. Unlike imaging markers in the prior art, the interstitial marker provided in this invention is reliably visible under each one of X-ray, US and MRI (that is, the same marker will be visible in X-ray, US and MR systems). In MRI systems, the marker exhibits MR susceptibility that can be controlled so that a signal void is produced in spin-echo or gradient echo MR imaging sequences and serves to outline the marker in its true position. The interstitial marker also achieves optimal reflectivity for US contrast independent of its orientation and placement in the body, thereby yielding reliable acoustic shadowing identification regardless of the relative orientation of the US probe to the marker geometry. The interstitial marker also exhibits sufficient X-ray opacity to be visible under X-ray images and CT scans due to its constituent components. The iron may be provided to enhance the MR susceptibility of the system, and the iron may be present in the glass or aluminum microspheres or as a distinct additive in the gelatin, as spheres or particles. The term particles includes both solid and hollow particles, but as noted later in the discussion with respect to acoustic properties of the spheres with respect to ultrasound, all particles should not be with sufficient absorption characteristics as would absorb ultrasound to a degree as to reduce its effectiveness.

Viewed from another aspect, the present invention provides a method for altering the composition of the imaging marker to enable the incorporation of a number of diverse contrast generating materials. Selection of a small microsphere volume relative to the gel volume ensures that adequate gel material is available in the marker volume to provide mechanical stability and microsphere binding. In addition, the gel provides a substrate of sufficient volume to add various contrast generating materials, such as, for example, water soluble paramagnetic species and fluorescent material. In a preferred embodiment, an optical fluorophore can be added to the gel for optical detection. A non-limiting example of such a fluorophore is indocyanine green, which strongly binds to proteinaceous substrates and has recently been approved by the FDA for human use. In another preferred embodiment, optical markers such as quantum dots can be added to the composition of the marker to provide bright optical emissions, as previously reported in the medical literature by West (West J L., 2003 Ann Rev Biomed Eng 5: 285-93).

A further alternative distinguishing feature of the technology described herein is that placement of the localization marker may be entirely interstitial. This aspect of the technology allows the tumor localization procedure and surgery to be carried out in separate stages, when this is appropriate in terms of the patient's health status and related medical factors. Although the marker was initially developed for tumor localization in image guided breast surgery and biopsy procedures, it is also useful for numerous other diagnostic procedures, such as MR spectroscopy, carried out under imaging guidance in breast or other areas of the body.

One aspect of the presently described original technology is to provide an MRI, US and X-Ray imaging compatible marker for improved localization of tumors and other tissue abnormalities.

Another aspect of the presently described original technology is to provide an implantable imaging marker with stable and reliable imaging characteristics on MRI, US, and X-ray that is useful for pre-operative and intra-operative surgical guidance, as well as post-operative monitoring.

Yet another aspect of the presently described original technology is to provide a small tissue-compatible marker device that can be inserted through the biopsy needle at the time of biopsy, thereby providing a radiographic target for future localization in the event of surgery.

A further aspect of the presently described original technology is to provide a method wherein the composition of the imaging marker can be altered using microspheres to incorporate paramagnetic and ferromagnetic materials yielding desirable proton density, T1 relaxivity and T2 susceptibility characteristics on MRI.

Another aspect of the presently described original technology is to provide a method wherein the composition of the imaging marker can be further altered using microspheres to achieve optimal US reflectivity.

Yet another aspect of the presently described original technology is to provide a method wherein the composition of the imaging marker can be altered by adding an optical fluorphor in order to generate optical contrast for intra-operative visibility to a relatively shallow depth under infra-red excitation.

These and other features, aspects, and advantages of the present invention will be apparent upon consideration of the figures and the following detailed description of the presently described original technology.

### BRIEF DESCRIPTION OF THE FIGURES

FIG. 1 shows both (a) Schematic diagram of marker composition. (b) Photograph of a marker containing 180 microspheres bound in a gel matrix.

FIG. 2 shows images of US-guided marker delivery. (a) The insertion cannula containing the marker at its tip. (b) A magnified view of the tip of cannula containing the marker. (c) An illustration of how the marker is inserted into the chicken breast under US guidance. (d) The corresponding US image shows the insertion of cannula (arrowheads) containing the marker at the tip (arrow) inside the breast tissue.

FIG. 3 shows images in a phantom containing 3 microspheres made of different materials with the corresponding US image (a) and the US echo intensity distribution along the line joining the three microspheres (b).

FIG. 4 shows a US image of single glass microsphere (arrow) in a chicken breast (a) and the corresponding echo intensity plot along the depth of single microsphere (b). The US image of a collection of 10 glass microspheres (arrow) in the same tissue (c) and its echo intensity plot along the depth of 10 microspheres (d).

FIG. 5 shows US images of 1.42 mm markers with 10%, 40% and 90% glass mass concentration in a phantom (a) and the normalized peak US intensity for different glass mass concentration (b).

FIG. 6 shows US images of a chicken breast tissue containing the 2.05 mm marker of 40% mass concentration in the axial orientation (a) and sagittal orientation (b).

FIG. 7 shows a US image of markers of different size containing 40% glass microsphere mass concentration in a chicken breast tissue.

FIG. 8 shows an axial MRI of 2.05 mm markers iron content range from 0 μg to 468 μg in separate phantoms (a). The image was acquired at 1.5 T using surface coil with a 2D SPGR sequence TR/TE/FA=18.4 ms/4.2 ms/30°. The average size of the imaging void as a function of iron content for two different TE values (b) is provided. Imaging was performed with 2D SPGR sequence TR/FA=18.4 ms/30° (o, TE 4.2 ms; *, TE 7.3 ms).

FIG. 9 shows axial (a) and sagittal (b) MRI of the final marker which was placed parallel to B₀ in phantom. Axial (c) and sagittal (d) MRI of the same marker which was placed perpendicular to B₀. Imaging was done with a 2 D SPGR sequence TR/TE/FA=18.4 ms/4.2 ms/30°.

FIG. 10 shows MRI (a), US image (b) and X-Ray image (c) of the final marker in a chicken breast tissue.

### DETAILED DESCRIPTION OF THE INVENTION

X-ray mammography remains the primary screening and initial detection method for breast cancer. The distinction between benign and malignant masses is generally made by analysis of the margins, shape, density,₁₅ analysis of the margins, shape, density, and size of any detected lesion. A benign lesion, such as a cyst or fibroadenoma, typically has a sharply circumscribed margin and oval or round shape, whereas malignant masses often exhibit speculated contours due to the infiltrative nature of breast cancer. However, mammography has significant limitations in terms of imaging sensitivity and specificity.

MR imaging has become a viable adjunct to X-ray mammography for detecting breast lesions. Some reports indicate that MRI can yield 100% sensitivity in the detection of malignant breast lesions. Using contrast enhanced MR imaging methods, malignamt and benign tumors that cannot be seen with mammography are visible on MR images. Furthermore, by incorporating a number of morphologic breast lesion characteristics, the specificity of MRI detection of breast lesions has increased significantly. The architectural features which have been found to be most useful in characterizing MR-visible breast lesions include lesion border irregularity and non-uniform lesion enhancement. Conversely, smooth bordered or lobulated lesions or non-enhancement have been found to be predictive of benign lesions. Morphologic assessment of breast lesions requires high spatial resolution contrast-enhanced 3D MR. Such high-resolution visual images can be extremely useful to the clinician in pre-operative planning. Imaging localization markers, such as interstitial marker disclosed in the present description of original technology that are all of MRI, X-ray and US-visible, and can be dynamically monitored by each three imaging modalities, are likely to have considerable utility in pre- and intra-operative surgical and biopsy procedures.

In many cases, it is necessary for a surgeon to pre-operatively localize abnormal tissues that are to be resected in a subsequent operative procedure. Precise localization of tissue is also required during biopsies because the biopsy site must be reproducible in the event further biopsy or surgery is required. To facilitate localization of such tissue sites, markers are temporarily inserted into the tissue at the required location. When a needle biopsy of a breast lesion lacks clear radiographic evidence of the extent of the tumor because of insufficient image contrast between normal and abnormal tissue or as a result of image distortion caused by imaging artifacts, pre-operative planning is difficult. Furthermore, when excisional biopsy results suggest cancer, further localization may be carried in order to plan for further surgical resection of the tumor bed. Thus, if radiographic definition of abnormal tissue is unclear, subsequent localization is problematic.

Most prior art methods for localizing breast lesions involve the use of a hypodermic needle placed into the breast in close anatomic proximity to the lesion. The hypodermic needle is withdrawn over a wire and the wire anchored until after surgery. However, compression of the breast during mammographic filming can cause the wire to move or be displaced with respect to the breast lesion. Several patents, such as U.S. Pat. No. 4,592,356 (Gutierrez), U.S. Pat. No. 5,059,197 (Urie et al.), U.S. Pat. No. 5,127,916 (Spencer et al.), U.S. Pat. No. 5,800,445 (Ratcliff et al.) and U.S. Pat. No. 5,853,366 (Dowlatshahi) disclose the use of various straight, curved or helical localization devices having an anchoring component at a distal end to firmly anchor the device into the tissue. However, such prior art markers cannot be left in the patient's body for future image-guided procedures, and typically are removed within a short period after insertion.

Historically, markers used in interventional and surgical procedures have often been made of radiopaque materials so that their precise location could be identified through X-ray viewing. X-ray opaque materials are disclosed in the prior art and can take the form of radio-opaque resins, or other similar compositions such as disclosed in U.S. Pat. No. 4,581,390 (Flynn) or barium, bismuth or other radio-dense salts, such as disclosed in U.S. Pat. No. 3,529,633 to Vaillancourt and U.S. Pat. No. 3,608,555 (Greyson). Similarly, X-ray markers may be formed of metal such as platinum, as disclosed in U.S. Pat. No. 4,448,195 (LeVeen). Exemplary of guidewires markers used under X-ray viewing is the invention disclosed by U.S. Pat. No. 4,922,924 (Gambale et al.).

More recently, imaging markers have been developed that are visible on MRI. For example, U.S. Pat. No. 5,375,596 (Twiss et al.) discloses a method for locating tubular medical devices implanted in the human body using an integrated system of wire transmitters and receivers. U.S. Pat. No. 4,572,198 (Codrington) additionally provides for conductive elements, such as electrode wires, for systematically disturbing the magnetic field in a defined portion of an interventional device to yield increased MR visibility of that region of the device. However, the presence of conductive elements in the imaging device also introduces increased electronic noise and the possibility of Ohmic heating, and these factors have the overall effect of degrading the quality of the MR image and raising concerns about patient safety. Thus, the presence of MR-incompatible wire materials in implantable medical markers disclosed in the prior art causes large imaging artifacts on MRI. Lack of clinically adequate MR visibility and/or imaging artifact contamination caused by the device is also a problem for most commercially available catheters, microcatheters, shunts, and other probes that can be used with image-guided methods.

The limitations inherent in imaging markers disclosed in the prior art have led to explorations of alternative tumor marking techniques. The ideal marker for tumor localization would be entirely interstitial to allow the patient to return home after the localization procedure without compromising the patient's outcome. Furthermore, the marker may need to be left in a precise location in the tissue for long periods to facilitate the investigation of lesions that require serial imaging over a period of weeks or perhaps months. Thus, it would be desirable to anchor the interstitial marker so that the device does not migrate from its insertion site in tissue. A number of mechanical anchors disclosed in the prior art, for example in U.S. Pat. No. 4,592,356 (Gutierrez,) U.S. Pat. No. 5,059,197 (Urie et al.), U.S. Pat. No. 5,127,916 (Spencer et al.), U.S. Pat. No. 5,800,445 (Ratcliff et a.), U.S. Pat. No. 5,853,366 (Dowlatshahi) and U.S. Pat. No. 6,181,960 (Jensen et al.) could be used. More preferred is the use of a fixative, such as the fibrogen-based adhesive described in multiple references in the medical literature, for example, Alam et al (Alam H B, et al., 2005 Mil Med 170: 63-9), Katkhouda (Katkhouda N, 2004 Surg Technol Int 13: 65-70), Kraus et al. (Kraus T W, et al., 2005 J Am Coll Surg 200:418-27), Singer et al. (Singer M, et al., 2005 Dis Colon Rectum), and Uy et al. (Uy H S, et al., 2005 Ophthalmology 112:667-71). Also preferred is the use of an autologous fibrin, such as described by Hirayama et al (Hirayama T, et al., 2005 Kyobu Geka 58:128-32), which could be used as a ‘glue’ to effectively ‘cement’ the interstitial marker at a specific tissue location.

According to the original technology described herein, the interstitial marker should also be made of sterilizable material that is mechanically and chemically stable and of low thrombolytic and inflammatory potential when implanted in tissues. Sterility of the marker can be achieved using coating procedures employing biocompatible membranes as described in the prior art. Examples of biocompatible materials which could be used to practice the present invention include elastin, elastomeric hydrogel, nylon, teflon, polyamide, polyethylene, polypropylene, polysulfone, ceramics, cermets steatite, carbon fiber composites, silicon nitride, and zirconia, plexiglass, and poly-ether-ether-ketone.

In accordance with the original technology described herein, the marker should exhibit high contrast in all relevant imaging methods including X-ray, US and MRI. Imaging markers used under MR guidance should also be MR-compatible in both static and time-varying magnetic fields. Many materials with acceptable MR-compatibility, such as ceramics, composites and thermoplastic polymers, are electrical insulators and do not produce artifacts or safety hazards associated with applied electric fields. Some metallic materials, such as copper, titanium, brass, magnesium and aluminum are also generally MR-compatible, such that large masses of these materials can be accommodated within the imaging region without significant image degradation. In one preferred embodiment, the interstitial marker of the present invention can be made MR visible by doping the marker with a material which has an MR resonance based on ¹⁹Fluorine. ¹⁹Fluorine-labelled materials have been used previously for MRI studies of tissue oxygenation (Mason R P, et al., 2003 Adv Exp Med Biol 530:19-27) and metabolism of L-DOPA (Dingman S, et al., 2004, J Immunoassay Immunochem 25:359-70), as well as to track uptake of 5-Fluorouracil (Klomp D W, et al., 2003 Magn Reson Med 50: 303-8). In a particularly preferred embodiment of the presently disclosed original technology, the interstitial marker can be clearly visualized on the basis of the ¹⁹Fluorine resonance in a clinical 1.5 Tesla MRI scanner by employing dual tuned transmit/receive coils set at 60.08 MHz for Fluorine and 64.85 MHz for protons, and using sequential or interleaved imaging of both resonances. By simply overlaying the resulting Fluorine and proton-based images, the location of the marker can be precisely determined in relation to contiguous tissues.

According to a method according to the original technology disclosed herein, providing a large gel volume in the marker allows a number of different contrast generating materials to be incorporated in the composition of the marker, including as two non-limiting examples, soluble paramagnetic and fluorescent material. Particularly preferred as a paramagnetic contrast agent is Gadolinium, which induces an increase in T1 relaxivity yielding increased signal on T1 weighted MRI. In another preferred embodiment of the invention, an optical fluorophore can be added to the gel for optical detection. A non-limiting example of such a fluorophore is indocyanine green, which strongly binds to proteinaceous substrates and has recently been approved by the FDA for human use. This fluorophore is excited by infra-red (805 nm) and generates a fluorescence in a slightly lower energy infra-red band (850 nm). In another preferred embodiment, optical markers such as quantum dots can be added to the composition of the marker to provide bright optical emissions, as previously reported in the medical literature by West (West J L., 2003 Ann Rev Biomed Eng 5: 285-93).

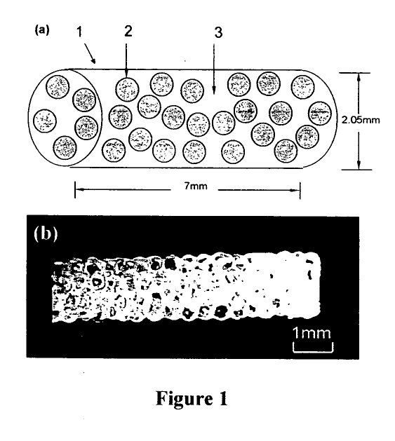

The method of the presently disclosed technology will now be described further by way of a detailed description of ex vivo studies with particular reference to certain non-limiting embodiments and to the accompanying drawings in FIGS. 1 to 10.

It is also important to appreciate the conventional bases upon which the characteristics of image quality are usually considered within each of the three imaging technologies, ultrasound, Magnetic Resonance and X-ray.

X-Ray Properties.

X-rays in the diagnostic energy regime are absorbed in materials principally on the basis of their electron density and atomic number and vary as a function of x-ray energy. Biological tissues are very similar to water in their attenuation properties for X-rays. The goal for an x-ray marker is that it should exhibit an attenuation coefficient sufficiently different from that of tissue to be observable in typical image capture systems (e.g., CCD, photography, photohtermography, or other electronic/optical detection systems). These differences could be exhibited as either a smaller or larger attenuation to x-ray, as long as they differ sufficiently from that of water as to provide the visible or detectable variation in properties. Tissues in general exhibit a relatively low attenuation coefficient, so selecting a marker of a material of high attenuation coefficient as the candidate materials could be considered as the simplest approach. Referring to Table I, it is seen that the linear attenuation coefficient for tissue is 0.72 cm²/gm and 0.197 cm²/gm at 20 KeV and 60 KeV respectively. These two energies have been selected as they reflect a range of photon energies which span a typical monoenergetic equivalent energy range of diagnostic x-ray spectra from a mammographic (20 KeV) to an energy used for computed tomography (60 Kev). The practice of the claimed invention is not limited to this range, as it has been selected solely for the purpose of enabling and exemplifying a generic concept of the scope of the disclosed technology. The point is that the attenuation coefficient should be different, and by way of non-limiting examples, at least 5%, at least 10%, at least 15%, at least 20%, and at least 25% different from that of water. This difference could be either higher or lower than the attenuation coefficient of water, although it is generally easier to select and work with materials having higher attenuation characteristics than that of water. Thus the X-ray marker may comprise a material which falls outside this range shown as the “hi’ and “lo” variants on the x-ray attenuation at each energy. That is an attenuation of less than 0.648/0.177 cm²/g at 20/60 KeV or more than 0.792/0.2167 cm²/g at 20/60 KeV, respectively. One can see that the materials glass, ceramics, metals (especially copper and aluminum) all meet this requirement. Of course these are just the obvious, non-limiting examples, and any solid or gelled material that exhibits this attenuation property may be used, such as composited, glasses, ceramics, metals, alloys, metal oxides, polymers, loaded or filled polymers, and the like. Many ceramics, other metals and plastics also meet this condition.

TABLE I
Properties of various candidate materials for the marker
X-ray, Acoustic and Magnetic Properties of Candidate Materials

| | | | | | X-ray | |
|---|---|---|---|---|---|---|
| | | speed | | | Attenuation | |
| | | of | acoustic | Magnetic | Coef (cm2/g) | |
| | density | sound | impedance | Suscepibility | 20 | 60 |
| Material | KG/m\hat{ }3 | (m/s) | (MRayl) | 10\hat{ }6 | KeV | Kev |
| glass | 2500 | 5640 | 14.1 | −13.8 | 2.3 | 0.241 |
| copper | 8940 | 3560 | 31.83 | −9.63 | 33.7 | 1.6 |
| aluminum | 2700 | 5100 | 13.77 | 20.7 | 3.44 | 0.277 |
| water | 1000 | 1493 | 1.49 | −9.05 | 0.72 | 0.197 |
| hi | | | 1.639 | −18.1 | 0.792 | 0.2167 |
| lo | | | 1.341 | −4.525 | 0.648 | 0.1773 |

Acoustical Properties:

Now with regard to the acoustical properties of the materials measured in ultrasound imaging, it is desirable to have a number of criteria satisfied. First, the materials should exhibit a difference in their acoustic impedance, which is in turn related to the material density and the speed of sound through the material. Referring to water as a surrogate for tissue, this means that we would like the material to exhibit values beyond the “hi” and “lo” values of impedance. Again, this is easily met by the non-limiting examples of candidate materials. Again, other materials such as ceramics, metals and some plastics could also be appropriate if they satisfy these constraints.

Another set of desirable properties for the acoustic marker materials is that they be particulate in nature, with such reular or irregular geometric shapes such as spherical, oval, rectangular, square, polyhedral, etc. in shape. They do not have be spherical or even, but it is desirable that they are not a flat or plate-like structure, as they should be readily observable from three dimensions. The idea is to make the internal reflectivity of the marker components look “rough” or bumpy with respect to the wavelength of the ultrasound we are considering. So, therefore one could use spheres, rough particles, grains, etc. They do not need to be all the same, but they should have reasonable projection areas when viewed from most if not all perpsctives, which is why the sphere or other form with three relatively large dimensions (e.g., a square or equilateral polyhedron) is useful. They could be random in their shape as long as they are closed (e.g., not having openings that would capture soundwaves), particulate-like, objects of approximately the same size. This will provide them with good acoustic scattering properties. This also suggests that the particles should be similar in size relative to the ultrasound wavelength. Thus if the particle were not larger than 10 times the wavelength they would still function well. Similarly, it is not desirable for a given wave for the particles to be too small relative to the wavelength. A reasonable relative size would be to keep them no less than 10% of the acoustic wavelength. Table II shows the corresponding wavelength in tissue for diagnostic ultrasound systems ranging from frequency of 5-15 MHz, which spans the current diagnostic ultrasound regime of interest. Again, the examples and displayed values are examples of a generic concept and are not intended to limit the disclosed practice of the present technology. The Table II also shows estimates of the most reasonable upper and lower bound for particle sizes based on these wavelengths in tissue.

TABLE II
Acoustic wavelength and Particle size limits

| | Frequency (MHz) | | |
|---|---|---|---|
| | 5 | 10 | 15 |
| Wavelength (mm) | 0.31 | 0.155 | 0.10 |
| Min particle size (mm) | 0.031 | 0.0155 | 0.01 |
| Max particle size (mm) | 3.1 | 1.55 | 1.0 |

Between the material acoustic properties (impedance) and size parameters, domains of values for selecting these particles have been generically characterized.
Magnetic Properties

The next factor to consider are the magnetic properties of the tissue and reference is again made to Table I. In this case, the characteristic reviewed is having the particles (e.g., the non-limiting examples of spheres are discussed) of essentially neutral magnetic susceptibility. In this case, it is desired to control the susceptibility of the marker as a whole by adding a small number of spheres of controlled levels of ferromagnetic impurity. Thus the majority of the spheres should be as close to tissue in terms of their magnetic susceptibility compared to tissue. Ideally the closer the better but anything within either 2 fold higher or lower would be acceptable. Glass particles were used, but it is clear that copper might even be better when it comes to controlling the susceptibility of the particles and minimizing susceptibility artifacts. Then by adding other spheres, such as the Aluminum spheres which contained some iron, controlled introduction of amounts of ferromagnetic doping to create a susceptibility artifact in gradient recalled images can be accomplished. In the studies, a range was explored of Fe from 0 μg to 460 μg and the effect was clearly observable. Thus, it is suggested that this is at least one example of a useful range of acceptability as a marker. The materials within this range were effective in each case. Any more than 460 μg would not necessarily be more helpful.

An alternative approach to the evaluation or characterization of this property associated with MR determinations would be to use a paramagnetic contrast agent which will cause T1 shortening. A good case in point, for a specific example of the generic class of materials recognized as MR contrast or marking agents would be to add Gd-DTPA to the gel formulation as it is water soluble. This can be characterized by the relaxivity of Gd-DTPA at 1.5 Tesla which is ˜4.5 sec⁻¹ mmol⁻¹. Thus the Gd-DTPA may be added to the volume of the gel, which is assumed to have the T1 of water. This would be the case as long as the particles do not exhibit large susceptibility changes. So, in this case, a formulation with copper might be better as it is very close to the susceptibility of water, and it will not create sizeable signal voids. Then by adding Gd-DTPA, the T1 of the gel marker can be shortened. The amount of Gd-DTPA required depends on the tissues in which the marker will be placed and how bright (how significant a contrast) is desired from the marker. For example, if the goal is to use the marker in breast tissue, the T1 of the native tissue is ˜0.7 seconds at 1.5 Tesla. Now, it would be desired to have the marker display at least a 10% difference in the relaxation characteristics. So, the gel would be doped so that the gel plus marker would have a T1 less than 0.7 seconds (at least in those areas of the marker that have been doped, to give a postive contrast in the final image. The actual concentration or weight amount of the marker is again dependent upon the specific results desired and the tissue to which it is applied. It is estimated that at least a 10% reduction in T1 would be desirable, but the larger the difference the better. So, it could be suggested to reduce this T1 of the tissue in this case to 0.63 seconds for at least modest visability on T1 weighted MRI at 1.5 Tesla. This can be easily calculated on the basis of the relaxivity of the contrast media using the following formula;

$$\frac{1}{T 1} = \frac{1}{T {1}_{0}} + R 1 [ Gd ]$$

Were T1₀ is the T1 of the gel matrix of the gel without any Gd-DTPA included, R1 is known as the T1 relaxivity of Gd-DTPA and [Gd] is the concentration of the Gd-DTPA in the gel solution. The T1 for 1.5 Tesla is 4.5 sec⁻¹ mmol⁻¹. The basis of measurements can also be determined at other MR field intensities such as 2.0 Tesla, 2.5 Tesla, 3.0 Tesla and even higher, but whatever the intensity of the field, the objective is to provide a detectable signal change between the tissue and the marker that is useful to the practitioner

Marker Fabrication.

In one embodiment of the original technology disclosed herein, the interstitial marker is preferably comprised of small microspheres suspended in a gelatin matrix. By appropriate selection of materials, optimal marker visibility can be produced in a single device for all of and each of MRI, US and X-Ray applications. In another preferred embodiment, the composition of the marker exhibits a density and an average atomic number of the tissue. Tissue is composed of nitrogen, carbon, oxygen, hydrogen, etc. These all have differing atomic numbers so that an average atomic number depends on their relative abundance in the particular tissue in which the marker is placed. Very roughly, tissue can be considered as a hydrocarbon and its “atomic number” would be somewhere near 6-7, but would be higher in bone, which would be composed of calcium as well, thus raising the avegage atomic number. If the marker is made out of aluminum, silicon or copper, the atomic number of the marker is much higher than those constituents for tissue. These materials would have an effective atomic number that is substantially higher than those of tissue to ensure X-Ray visibility. In a further preferred embodiment of the technology disclosed herein, the composition of the marker has a substantially high acoustic impedance difference from the surrounding tissue to provide good US contrast. In yet another preferred embodiment of this invention, the magnetic susceptibility of the marker is similar to that of tissue in order to control MRI contrast in T2* weighted images.

Table 1 summarizes a number of desirable physical properties of glass, copper and aluminum, as three non-limiting examples of materials that could be used to produce the interstitial marker according to the present invention. The magnetic susceptibilities of these materials are all reasonably close to that of tissue but additionally can include controlled doping with ferromagnetic or paramagnetic materials selected for particularly desirable T1 and T2 properties on MRI. The ferromagnetic and paramagnetic agents can be incorporated as aqueous solutions or suspensions. By way of example, the paramagnetic materials selected can include transition metal ions such as gadolinium, dysprosium, chromium, nickel, copper, iron and manganese, or stable free radicals such as nitroxyls. The concentration of the paramagnetic agents can range from the micromolar to millimolar range. Non-paramagnetic materials having desirable MR relaxation characteristics may also be employed in the manner set forth above to practice the present invention.

With regard to the X-ray properties of the selected glass, copper and aluminum materials, it was found that the materials exhibit a 3.2-46 fold increase in total X-ray absorption coefficient compared to water at an energy equivalent to a mammographic exposure (˜20 KeV) (Plechaty E F, et al., 1978 Lawrence Livermore National Laboratory Report UCRL-5400). Similarly, the density and speed of sound in these materials was found to result in an 11-24 fold increase in acoustic impedance compared to that of water (Krautkramer J and Krautkramer H, 1990 Ultrasonic Testing of Materials, Springer Verlag, ISBN: 0387512314), thus ensuring good US reflectivity.

In accordance with a preferred embodiment of the invention, the bulk of the marker is comprised of glass microspheres, which are readily available, biocompatible and provide all required features for optimal US and X-Ray contrast. Particularly preferred are GL-0175 glass microspheres (MO-SCI Corporation, 4000 Enterprise Drive, Rolla, Mo. 65402, USA) in diameters ranging from 0.4-0.6 mm with a density of 4.2-4.5 g/cm³. Also preferred are aluminum microspheres (Salem Specialty Ball Corporation, West Simsbury, Conn. 06092, USA) 0.5 mm in diameter with small amounts of iron (0.7% by mass) making them slightly ferromagnetic. In a further preferred embodiment of the invention, it was found that adding a small number of iron doped aluminum microspheres to the marker reliably induces a small but detectable B₀ inhomogeneity around the marker which presented as a signal void in T2* weighted MRI. As an alternative non-limiting embodiment, it was also found that pure copper microspheres of 0.8 mm in diameter (Salem Specialty Ball Corporation, West Simsbury, Conn. 06092, USA) could be used instead of glass microspheres.

In a further non-limiting embodiment of the original methods of this idsclosure, the aluminum and glass microspheres were suspended in a 10% gelatin solution (Sigma Chemical Corporation, 3050 Spruce Street, Saint Louis, Mo. 63103, USA) (FIG. 1(a)). The gelatin mixture was prepared by mixing with distilled water at 85-95 degrees Celsius. The glass and aluminum microspheres were then added in the correct numbers to achieve significant Ultrasound response and the mixture was cast in a 12-gauge needle. The mixture was allowed to cool at room temperature for 2 hours and then refrigerated at 4° C. for another 24 hours. With reference to FIG. 1, upon completion of cooling, the marker was semi-rigid and could be removed from the needle mold in the form of a cylindrical structure 1, 7 mm long with 2.05 mm diameter containing the microspheres 2 and gelatin 3. FIG. 1(b) is a photograph of the final form of the marker suitable for delivery with a 12-gauge biopsy needle that is routinely used clinically for breast tumor localization.

In accordance with the original method disclosed herein, the imaging contrast of the marker for MRI visualization was controlled by adding a variable number of iron-containing aluminium microspheres to the marker corresponding to an iron content from 0 μg to 468 μg. The US contrast was modulated by adjusting the number of glass and aluminium microspheres added to the gelatin matrix. The optimal mixture was determined to provide maximum US contrast while providing clear localization of the marker in MRI and mammography.

Imaging validation studies were performed with either homogeneous agar phantoms or ex-vivo tissue samples. The phantoms were prepared with agar (Sigma Chemical Corporation, 3050 Spruce Street, Saint Louis, Mo. 63103, USA) and distilled water. Amorphous silica powder (Sigma Chemical Corporation, 3050 Spruce Street, Saint Louis, Mo. 63103, USA) was also added to provide the phantom with a background of US backscattering material to simulate tissues. Two kinds of homogeneous phantoms were prepared: the first kind of phantom was rectangular in structure (60×60×40 mm) and designed for the US contrast study; the second kind of phantom was cylindrical in structure (40 mm long and 30 mm in diameter) and used for the MRI contrast study. All of the phantoms were composed of 4% agar mixed with 4% silica. Tissue phantoms were used in the form of fresh chicken breast tissue. Three samples of chicken breast were used for the US study, while a piece of chicken breast containing a small segment of bone (12.6 mm long) was used for a comparative study of the marker with each imaging modality.

Ultrasound Imaging Studies

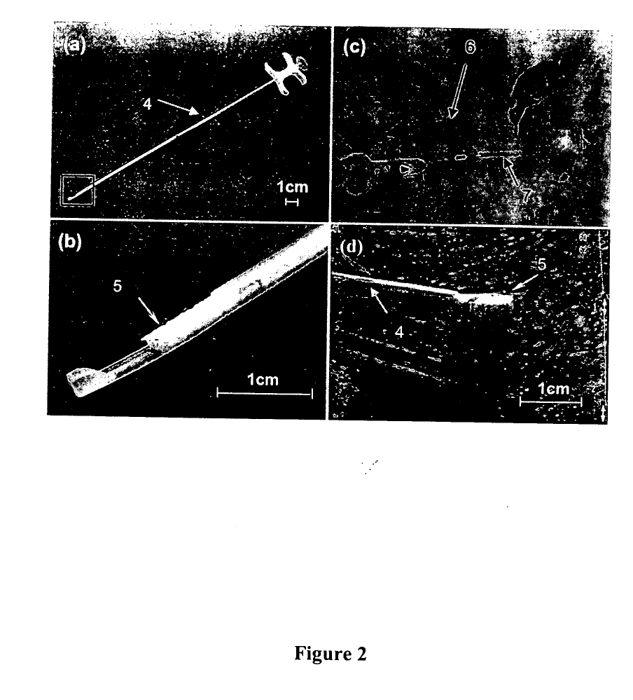

The markers were placed in the phantoms under US guidance using a Philips ATL HDI-5000 imaging system with a Broadband linear array 5-12 MHz transducer (L12-5 50 mm, Philips). With reference to FIG. 2, each marker was loaded into a 12-gauge blunt cannula 4 before placement. The marker 5 was placed in the tissue 6 by first using an 11-gauge co-axial introducer needle 7 with a trocar (MRI Devices Corporation) to form a path into the phantom. After positioning the introducer needle, the trocar needle was withdrawn and then a 12-gauge cannula 4 containing the marker was passed through the introducer needle, as shown in FIG. 2(c). In order to confirm the correct position of the cannula tip, US guidance was used before releasing the marker 5, as shown in FIG. 2(d). Finally, the marker 5 was left in the desired position by first pushing it out from the cannula 4 and then removing the cannula and introducer needle 7 from the tissue. Axial and sagittal US imaging was performed to verify the position of the marker. During US scanning, the gain and dynamic range were adjusted with the target placed at the focal zone to provide the best contrast. In order to measure the echogenicity of the markers, the US echo intensity was used on B-Scan images in orthogonal directions through the marker location. The peak echo signals were measured for each glass and aluminum microsphere concentration and normalized to the maximum echo signal.

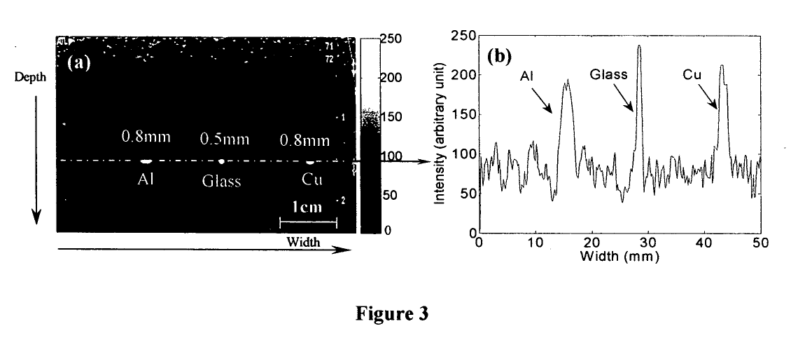

A series of phantom and in vitro tissue experiments were used to determine the optimum marker composition. The US image of a rectangular phantom injected with a single glass, aluminum and copper microsphere 8 is shown in FIG. 3 (a). The three microspheres were deposited at the same depth to ensure that the microspheres were exposed to the same acoustic conditions. The US echo intensity profile through the microspheres is shown by the dashed line in FIG. 3 (a) through each microsphere. It was found that although the glass microsphere was smaller than the aluminum or copper microspheres, they demonstrated a slightly greater signal than either the aluminum or the copper microspheres. Since the glass microspheres produced clearly defined US echoes and are biocompatible, they were chosen to form the bulk of the marker content in accordance with the method of the invention.

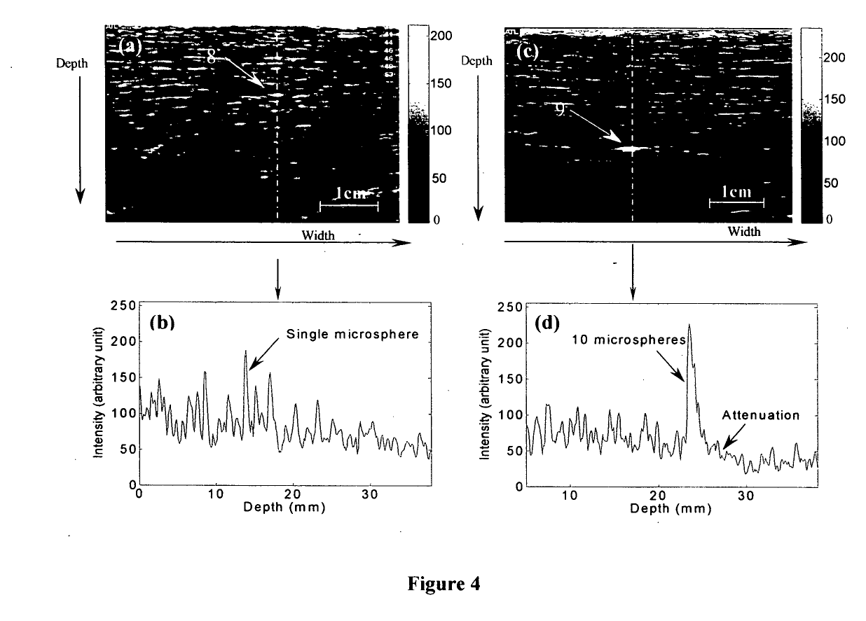

With reference to FIG. 4, in order to evaluate the effect of the number of glass microspheres on marker contrast, the US intensity for a single glass microsphere was compared to a collection of 10 microspheres injected into the same chicken breast 6. As shown in FIG. 4 (a), the single microsphere 8 is less well resolved. The intensity distribution along the depth of the single glass microsphere, as illustrated in FIG. 4 (b), is difficult to differentiate from the surrounding breast structure. By comparison, the collection of 10 glass microspheres 9 appears as a hyperintense structure with acoustic shadowing, as shown in FIG. 4 (c). With reference to FIG. 4 (d), the corresponding acoustic intensity distribution along the depth of 10 microspheres 9 shows a clear echo in the US data demonstrating a marked contrast improvement with the larger number of glass microspheres.

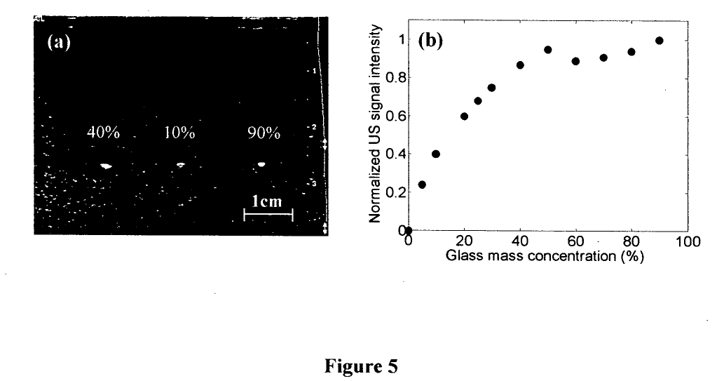

With reference to FIG. 5, to evaluate the effect of glass microsphere concentration suspended in the gel matrix, US intensity was measured in phantoms 10 with 1.42 mm markers of different glass concentrations. The US image of the three markers shown in FIG. 5 (a) demonstrates that a variation in the marker visibility results from different concentrations of glass microspheres. As described for the previous imaging study, the three markers were deposited in an agar phantom at the same depth for the same acoustic conditions. The effect of varying the ratio of glass microsphere volume to the total marker volume was studied using 2.3%, 8.4% and 20.7% compositions, corresponding to glass mass to total marker mass of 10%, 40% and 90% or using 3, 13 and 27 glass microspheres, respectively. The relative US peak echo intensity is plotted in FIG. 5 (b) as a function of glass mass concentration and shows that the optimal concentration should be greater than 40% weight by volume. In accordance with the method of the invention, it was found that a marker of 40% mass concentration occupied only 8.4% of the marker volume, thus providing a large gel volume to ensure solid binding of the spheres in the final marker.

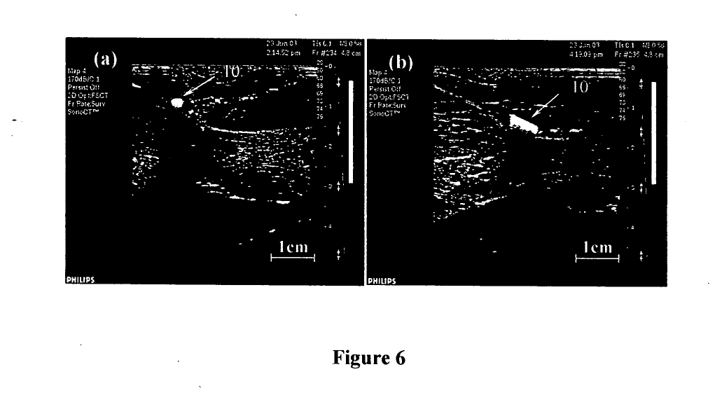

In accordance with the original technology disclosed herein, in order to aid in identifying the marker with US, a generally cylindrical shape (for example, one dimension such as length, being at least 1-%, at least 20%, at least 30% or at least 40% greater than each of the other two dimensions such as width and depth, and with the other two dimensions such as width and depth generally differing from each other by less than 50%, less than 40%, or less than 30% compared to the smallest dimension, and the cross-section may be circular, oval, triangular, rectangular, or other regular or irregular shapes) is preferred because it presents a predicable change in the appearance with different US orientations. Less preferred is a spherical, square, polyhedral or other geometric or irregular marker which may have a similar appearance from multiple imaging angles. This is illustrated in FIG. 6, where two orthogonal US views demonstrate how the cylindrical geometry of the marker aids in its unique identification.

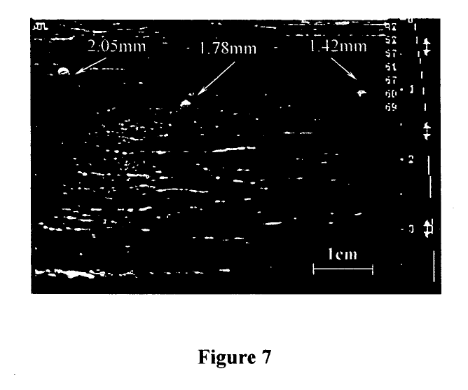

The results with different marker sizes are shown in FIG. 7, where the US image was obtained from markers with diameters of 1.42 mm, 1.78 mm and 2.05 mm injected into a chicken breast. In this case, the glass concentration of these markers is 40% by weight. All of the markers appear as bright circular structures and demonstrate that contrast increases with marker size. Thus, in accordance with the method of the invention, the 2.05 mm marker appears to provide a practical compromise between minimum invasiveness and good US visibility.

It has also been disclosed in the art that irregular surface particles, whether hollow or solid, can provide enhanced reflectivity of ultrasound, and such constructions are useful herein. (see Burbank et al., Published U.S. Patent Application No. 20050063908, which is incorporated herein by reference) Similarly, nanostructured surfaces of particles or spheres or other shapes may be used to enhance Ultrasound reflectivity (as described in Published U.S. Patent Application No. 20050038498, Dubrow et al., which is incorporated herein by reference).

MRI Studies

MR studies were performed on a 1.5-Tesla MRI system (Signa, G E Medical System) with a 5-inch surface coil and employing a standard 2D spoiled gradient recalled sequence (SPGR) clinical breast MRI protocol. The pulse sequence parameters were TRITE/FA=18.4 ms/4.2 ms/30°, with a bandwidth of 15.6 KHz and a spatial resolution of 0.39 mm in-plane and 2 mm slice thickness.

To measure the size of the MRI signal void resulting from markers with different iron content, four measurements along the horizontal, vertical and diagonal directions were performed for each marker. The width of the signal void was estimated between the peaks of the greatest absolute gradient of the signal surrounding the marker. This corresponded to the points of steepest descent on the artifact profile. The mean and standard deviation of the size of the signal void from the four directions was used to characterize the size of the signal void and its variability. The size of the signal void and its standard deviation were plotted as a function of iron content at two different TE values (4.2 and 7.3 ms).

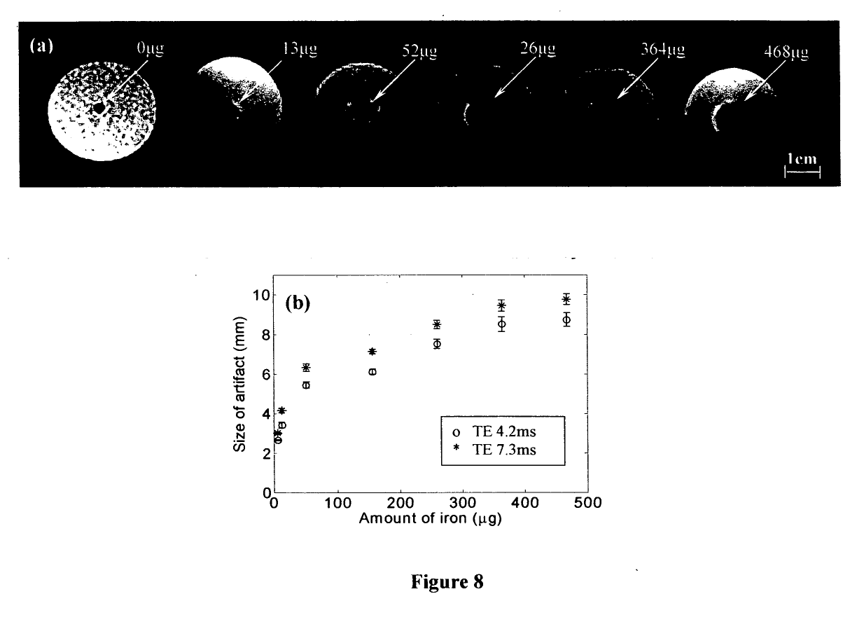

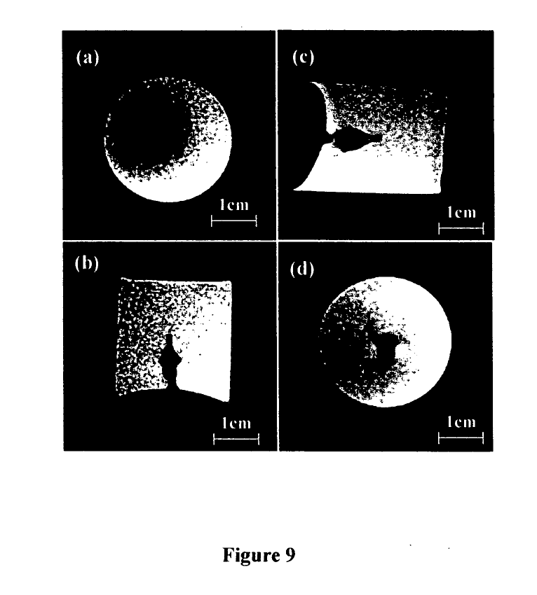

In accordance with the original technology disclosed herein, alternative compositions of the marker were evaluated in order to find the optimal iron content that allows clear marker definition on MRI without excessive distortion of the MR image from B₀ inhomogeneities. Accordingly, the effect of replacing some glass microspheres with the same number of iron-containing aluminum microspheres was tested. Imaging was carried with a gradient recall sequence (SPGR) at two different echo times as shown in FIG. 8 (a), with the direction of the axis of the marker parallel to B₀. It was found that increasing the iron content of the marker generated a larger imaging void. The size of the void was measured and plotted as a function of iron content as shown in FIG. 8 (b). The signal void was found to vary from 2.4 mm to 8.7 mm in diameter for a TE of 4.2 ms, and from 2.4 mm to 9.78 mm for a TE of 7.3 ms. A TE of 4.2 ms was chosen to comply with standard clinical breast MRI protocol. The results indicate that the marker containing ˜180 glass spheres and 52 μg iron produces a void artifact of 5.15 mm in diameter for a TE of 4.2 ms. This signal artifact is comparable to prior art studies in which MRI artifacts of 8 to 18 mm were produced by FDA approved stainless steel alloy clips (Meisamy et al 2004). However, it should be understood by those of ordinary skill in the art that MR contrast may be precisely controlled by adjusting the number, size, shape, and composition of the microspheres, as well as the MR imaging parameters. To evaluate the effect of the shape and orientation of the marker with respect to the magnitude of its susceptibility artifact, the axis of the marker was placed at different angles to B₀. With reference to FIG. 9, the axial 9 (a) and sagittal 9 (b) MR images showed that the marker appeared circular and rectangular when parallel to B₀. The sagittal image was somewhat irregular because of the local magnetic field inhomogeneity caused by iron. By comparison, when the marker was perpendicular to B₀, the axial 9 (c) and sagittal 9 (d) MR images of the indicated that the marker appeared oval and rectangular. This result demonstrated that the artifact of the marker is orientation dependent, in agreement with prior art studies (Seppenwoolde et al 2003).

X-Ray Imaging Studies

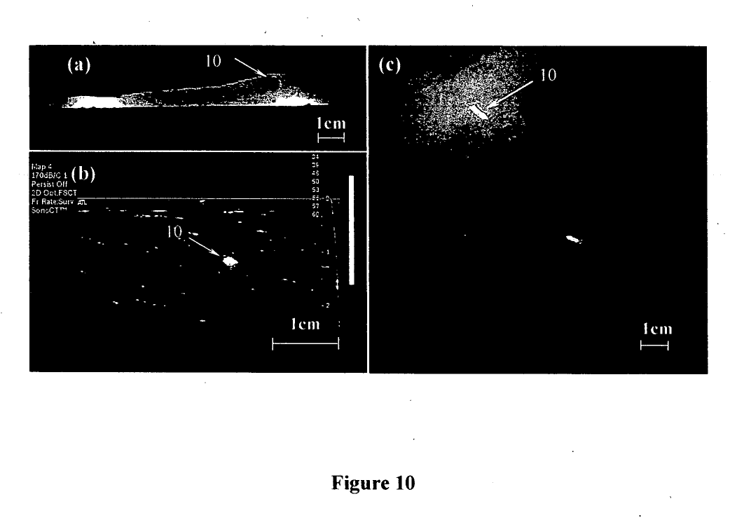

All X-Ray imaging studies were performed on a GE Senographe® 2000D full field digital mammography system using a tube voltage of 25 kVp, a tube current of 87 mA and a FOV of 13 cm. Modest compression was applied to the agar and tissue phantoms to simulate clinical conditions. With reference to FIG. 10, the image of the marker is seen as a region of increased X-Ray attenuation that exhibits sufficient X-ray opacity to make the marker visible under high quality X-ray images and particularly high resolution CT scans.

Comparative MRI, US, X-Ray Imaging Studies

The preceding imaging studies indicated that optimal MRI and US visibility is achieved with a marker diameter of 2.05 mm and 52 μg iron content. With reference to FIG. 10, the marker appears as a clear signal void on MRI 10 (a), while the US image of the marker shows a clear hyperintense structure with acoustic shadowing 10 (b). The X-Ray image clearly identifies the marker as a radio-opaque structure 10 (c). It is thus evident that this construction and composition of the imaging marker of the present invention is clearly visible under standard MRI, US and X-Ray examination

Although the presently disclosed original technology has been described mainly in terms of an imaging marker for localizing breast lesions, it will be understood by those of ordinary skill in the art that the availability of an interstitial marker visible on MRI, US, and X-ray, such as disclosed in this invention, would facilitate obtaining useful imaging information under all three imaging modalities in numerous surgical and interventional procedures. Medical and surgical applications of the invention would include vascular surgery and interventional radiology, cardiac surgery and cardiology, thoracic surgery and radiology, gastrointestinal surgery and radiology, obstetrics, gynecology, urology, orthopedics, neurosurgery and neurointerventional radiology, head & neck surgery and radiology, ENT surgery and radiology, and oncology. In addition to breast surgery and biopsy, the method of the invention applies to numerous interventional procedures that can be performed as intraluminal, intracavitary, laparoscopic, endoscopic, intravenous, and intra-arterial applications. A variety of probes, including surgical instruments, endoscopes, catheters, and other devices that can be inserted into the body can also be used with this invention.

Another general description of original technology described herein is provided by the following. An implantable image marker is provided for enabling non-invasive viewing of the marker subsequent to implantation. The marker may comprise a device with a surface (on or in the marker) of an artifact that has at least 10% difference in ultrasound reflectivity as compared to at least one of animal breast tissue, animal brain tissue, and animal heart tissue; a material that has at least 10% difference in relaxivity at the field strength use for MR imaging as compared to at least one of animal breast tissue, animal brain tissue and animal heart tissue, respectively; and a composition that has at least 10% difference in attenuation of X-rays from at least one of animal breast tissue, animal brain tissue, and animal heart tissue, respectively. By respectively, it is assumed that the marker will be implanted into approximately a single tissue composition, and that these differences should be evaluated with respect to that single tissue composition, and not to three different tissue compositions. The implantable marker may have at least two distinct particles supported in a matrix are used to provide the surface(s), the material that has at least 10% difference in relaxivity at 1.0 Tesla, and the composition that has at least 10% difference in attenuation of X-rays. The marker may be such that ultrasound reflectivity in the marker is provided at least in part by artifacts comprising particles exhibiting ultrasound reflectivity. A particularly good marker construction has ultrasound reflectivity in the marker provided at least in part by artifacts comprising particles exhibiting ultrasound reflectivity and the matrix comprises a gel. The exemplary particles comprise ceramic, glass, metal or metal oxide particles, and tthe particles may comprise ceramic, glass, metal or metal oxide particles and the surface of the particles comprise surface structure enhancing ultrasound reflectivity as compared to a particle of the same size and material having a smooth surface. Another construction comprises a material that alters MR relaxivity is present within a particle, such as a paramagnetic or superparamagnetic material selected from the group consisting of Cr, V, Mn, Fe, Co, Pr, Nd, Eu, Gd, Tb, Dy, Ho, Er, Tm, Tb and Ln. The composition for attenuation of X-ray may comprise at least one metal. One combination of particles (with similar or different shapes) may comprise a) a glass or ceramic particle and b) a metal particle. The marker may further comprise a fluorophore that emits detectible radiation when stimulated by electromagnetic radiation, current, or magnetic flux, preferably electromagnetic radiation (such as UV or IR radiation). In the use of particles, at least one particle may comprise aluminum particles comprises an iron content of >0 μg to 468 μg. The imaging marker may have a glass mass concentration greater than 40% weight by volume. The matrix or gel in said imaging marker may provide a substrate into which an MRI contrast agent can be added. The imaging marker appears as a clear hyperintense structure with acoustic shadowing on US images, and also appears as a radio-opaque structure on X-Ray images.

These particles may be used in a method of performing a medical procedure comprising identifying a region of treatment interest, implanting the marker described herein into tissue in that region of interest, subsequently viewing the region of interest and observing the location of the implanted marker by at least one of ultrasound, MR and X-rays, and performing a medical procedure on the region of interest identified by the marker. The subsequent viewing may be immediately thereafter, or at a later time such as at least 1 hour, at least 2 hours, at least 4 hours, at least 6 hours, at least 8 hours, at least 12 hours or at least 24 hours subsequent to implantation of the marker. Non-limiting examples of body regions where implantation of the marker may be provided include at least body regions of a patient selected from the group consisting of cardiovascular region, gastrointestinal region, intraperitoneal region, organs, kidneys, retina, urethra, genitourinary tract, brain, spine, pulmonary region, and soft tissues.

Surgical or treatment procedures such as invasive treatments or non-inavsive treatments may be used in combination with observation of the markers. Such treatments may be with surgical probe, catheter, or biopsy implements used to implants or position the marker, as well as pre-operative and intra-operative surgical guidance; localizing breast tumors under MRI, US and X-ray; excisional biopsy of the breast under MRI, US and X-ray; pre-operative localization procedures and surgery carried out on separate days; and any other local or target specific procedures. Examples of particular paramagnetic ions aere selected from the group consisting of Gd(III), Mn(II), Cu(II), Cr(III), Fe(II), Fe(III), Co(II), Er(II), Ni(II), Eu(III) and Dy(III), and a superparamagnetic agent may comprise a metal oxide or metal sulfide, particularly where the metal of the ion is iron. Other superparamagnetic materials may include ferritin, iron, magnetic iron oxide, manganese ferrite, cobalt ferrite and nickel ferrite. The implantable imaging marker may be made of material that is mechanically stable and tissue compatible, non-limiting examples being elastin, elastomeric hydrogel, nylon, teflon, polyamide, polyethylene, polypropylene, polysulfone, ceramics, cermets steatite, carbon fiber composites, silicon nitride, zirconia, plexiglass, natural or synthetic tissue, natural or synthetic gums or resins, sols and poly-ether-ether-ketone. The implantable imaging marker may be secured at its interstitial insertion site using a mechanical or chemical anchoring device. A chemical device would be an adhesive such as a fibrogen-based adhesive or an autologous fibrin. The implantable imaging marker may be made of sterilizable material that is of low thrombolytic/thrombogenic and low inflammatory potential when implanted in tissues. The materials may be coated for these or other effects at the site of implantation, including coatings or or diffusible material to effect those or other results, including local temporary pain or sensitivity reduction. To this end, sterility of said implantable imaging marker may be achieved using coating procedures employing biocompatible membranes. The implantable imaging marker may be MR-compatible in both static and time-varying magnetic fields.

In the preceding detailed description of the preferred embodiments, reference is made to the accompanying drawings which form a part hereof, and in which are shown by way of illustration specific preferred embodiments in which the invention may be practiced. These embodiments are described in sufficient detail to enable those skilled in the art to practice the invention, and it is to be understood that other embodiments may be utilized and that structural, logical, physical, computational, medical, architectural, and other related changes may be made without departing from the spirit and scope of the present invention. The preceding detailed description is, therefore, not to be taken in a limiting sense, and the scope of the present invention is defined only by the appended claims and their equivalents.

What is claimed:

1. An implantable image marker for enabling non-invasive viewing of the marker subsequent to implantation, the marker comprising a surface of an artifact that has at least 10% difference in ultrasound reflectivity as compared to at least one animal tissue a material that has at least 10% difference in relaxivity at a field strength used for magnetic resonance imaging as compared to at least one of animal breast tissue, animal brain tissue, and animal heart tissue, respectively, and a composition that has at least 10% difference in attenuation of X-rays from at least one of animal breast tissue, animal brain tissue, and animal heart tissue, respectively.

2. The implantable marker of claim 1 wherein at least two distinct particles supported in a matrix are used to provide the surface of an artifact that has at least 10% difference in ultrasound reflectivity as compared to at least one of animal breast tissue, animal brain tissue, and animal heart tissue, the material that has at least 10% difference in relaxivity at the magnetic resonance imaging field strength as compared to at least one of animal breast tissue, animal brain tissue, and animal heart tissue, respectively, and the composition that has at least 10% difference in attenuation of X-rays from at least one of animal breast tissue, animal brain tissue, and animal heart tissue, respectively.

3. The marker of claim 1 wherein ultrasound reflectivity in the marker is provided at least in part by artifacts comprising particles exhibiting ultrasound reflectivity.

4. The marker of claim 2 wherein ultrasound reflectivity in the marker is provided at least in part by artifacts comprising particles exhibiting ultrasound reflectivity and the matrix comprises a gel.

5. The marker of claim 3 wherein the particles comprise ceramic, glass, metal or metal oxide particles.

6. The marker of claim 4 wherein the particles comprise ceramic, glass, metal or metal oxide particles and the surface of the particles comprise surface structure enhancing ultrasound reflectivity as compared to a particle of the same size and material having a smooth surface.

7. The marker of claim 3 wherein a marker that alters MR relaxivityis present within a particle.

8. The marker of claim 7 wherein the marker that alters MR relaxivity is a paramagnetic materials selected from the group consisting of Cr, V, Mn, Fe, Co, Pr, Nd, Eu, Gd, Tb, Dy, Ho, Er, Tm, Tb and Ln.

9. The marker of claim 4 wherein a marker that alters MR relaxivity is present within a particle and the marker that alters MR relaxivity is a paramagnetic materials selected from the group consisting of Cr, V, Mn, Fe, Co, Pr, Nd, Eu, Gd, Th, Dy, Ho, Er, Tm, Tb and Ln.

10. The marker of claim 3 wherein the composition for attenuation of X-ray comprises at least one metal.

11. The marker of claim 4 wherein the composition for attenuation of X-ray comprises at least one metal.

12. The marker of claim 9 wherein the composition for attenuation of X-ray comprises at least one metal.

13. The marker of claim 11 wherein ultrasound reflectivity in the marker is provided at least in part by artifacts comprising particles exhibiting ultrasound reflectivity and the matrix comprises a gel.

14. The marker of claim 12 wherein ultrasound reflectivity in the marker is provided at least in part by artifacts comprising particles exhibiting ultrasound reflectivity and the matrix comprises a gel.

15. The marker of claim 1 comprising a) a glass or ceramic particle and b) a metal particle.

16. The marker of claim 4 comprising a) a glass or ceramic particle and b) a metal particle.

17. The marker of claim 11 comprising a) a glass or ceramic particle and b) a metal particle.

18. The marker of claim 14 comprising a) a glass or ceramic particle and b) a metal particle.

19. A method of performing a medical procedure comprising identifying a region of treatment interest, implanting the marker of claim 1 into tissue in that region of interest, subsequently viewing the region of interest and observing the location of the implanted marker by at least one of ultrasound, MR and X-rays, and performing a medical procedure on the region of interest identified by the marker.

20. A method of performing a medical procedure comprising identifying a region of treatment interest, implanting the marker of claim 2 into tissue in that region of interest, subsequently viewing the region of interest and observing the location of the implanted marker by at least one of ultrasound, MR and X-rays, and performing a medical procedure on the region of interest identified by the marker.

21. A method of performing a medical procedure comprising identifying a region of treatment interest, implanting the marker of claim 4 into tissue in that region of interest, subsequently viewing the region of interest and observing the location of the implanted marker by at least one of ultrasound, MR and X-rays, and performing a medical procedure on the region of interest identified by the marker.

22. A method of performing a medical procedure comprising identifying a region of treatment interest, implanting the marker of claim 9 into tissue in that region of interest, subsequently viewing the region of interest and observing the location of the implanted marker by at least one of ultrasound, MR and X-rays, and performing a medical procedure on the region of interest identified by the marker.

23. A method of performing a medical procedure comprising identifying a region of treatment interest, implanting the marker of claim 11 into tissue in that region of interest, subsequently viewing the region of interest and observing the location of the implanted marker by at least one of ultrasound, MR and X-rays, and performing a medical procedure on the region of interest identified by the marker.

24. A method of performing a medical procedure comprising identifying a region of treatment interest, implanting the marker of claim 24 into tissue in that region of interest, subsequently viewing the region of interest and observing the location of the implanted marker by at least one of ultrasound, MR and X-rays, and performing a medical procedure on the region of interest identified by the marker.

25. A method of performing a medical procedure comprising identifying a region of treatment interest, implanting the marker of claim 16 into tissue in that region of interest, subsequently viewing the region of interest and observing the location of the implanted marker by at least one of ultrasound, MR and X-rays, and performing a medical procedure on the region of interest identified by the marker.

26. A method of performing a medical procedure comprising identifying a region of treatment interest, implanting the marker of claim 4 into tissue in that region of interest, and after at least four hours subsequent to implantation of the marker, viewing the region of interest and observing the location of the implanted marker by at least one of ultrasound, MR and X-rays, and performing a medical procedure on the region of interest identified by the marker.

27. A method of performing a medical procedure comprising identifying a region of treatment interest, implanting the marker of claim 16 into tissue in that region of interest, and after at least four hours subsequent to implantation of the marker, viewing the region of interest and observing the location of the implanted marker by at least one of ultrasound, MR and X-rays, and performing a medical procedure on the region of interest identified by the marker.

28. The marker of claim 4 further comprising a fluorophore that emits detectible radiation when stimulated by electromagnetic radiation, current, or magnetic flux.

29. The marker of claim 4 wherein at least one particle comprises aluminum particles comprises an iron content of >0 μg to 468 μg.
